(19) United States
(12) Patent Application Publication
(10) Pub. No.: US 2009/0082812 A1
(43) Pub. Date: Mar. 26, 2009

(54) Provisional locking pedicle screw system and method

(76) Inventor: Trevor K. Lewis, Lehi, UT (US)

Correspondence Address: MINTZ LEVIN COHN FERRIS GLOVSKY & POPEO, ONE FINANCIAL CENTER, BOSTON, MA, 02111, US

(21) Appl. No.: 11/903,484

(22) Filed: Sep. 21, 2007

(51) Int. Cl. A61B 17/56; A61B 17/00; A61B 17/08

(52) U.S. Cl. 606/281; 606/103; 606/151; 606/301

(57) ABSTRACT

Provisional locking pedicle screw systems and associated methods for surgery are provided. A bone screw assembly may be provided that includes a bone screw, an anchor bracket, and a provisional locking member. The provisional locking member may exert a frictional force against the bone screw that semi-rigidly fixes the anchor bracket and the bone screw in place relative to each other. More rigid securement (e.g., non-provisional locking) of the relative positions of the bone screw and the anchor bracket may be achieved by securing a securement member (e.g., set screw) within the anchor bracket. Also provided are a multiple-component set screw and an anchor bracket configured to form a press-fit connection with the bone screw. In various embodiments, the systems and methods described herein may be used to fuse together, mechanically immobilize, and/or adjust the alignment of adjacent vertebrae of the spine.

### FIELD OF THE INVENTION

Embodiments of the present invention relate generally to systems and methods for spinal fixation, and more particularly, to provisional locking pedicle screw systems and associated methods for surgery.

### BACKGROUND OF THE INVENTION

The spinal column is a highly complex system of bones and connective tissues that provides support for the human body and protects the delicate spinal cord and nerves. The spinal column includes a series of vertebrae stacked one atop the other, where each vertebral body includes a relatively strong bone portion (i.e., cortical bone) forming the outside surface of the vertebral body and a relatively weak bone portion (i.e., cancellous bone) forming the center of the vertebral body. Situated between each vertebral body is an intervertebral disc formed from a non-bony, fibro-cartilage material that cushions and dampens compressive forces applied to the spinal column. The vertebral canal containing the delicate spinal cord and nerves is located just posterior to the vertebral bodies.

Various types of spinal column disorders are known and include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of the one vertebra over another, usually in a lumbar or cervical spine) and other disorders caused by abnormalities, disease or trauma, such as ruptured or slipped discs, degenerative disc disease, fractured vertebra, and the like. Patients suffering from these spinal disorders usually experience extreme and debilitating pain as well as diminished nerve function.

A technique commonly referred to as spinal fixation involves the use of surgical implants to fuse together, mechanically immobilize, and/or adjust the alignment of adjacent vertebrae of the spine. Such techniques have been used to treat the above-mentioned spinal column disorders. However, conventional spinal fixation devices have various disadvantages.

U.S. Pat. No. 5,474,555 describes a pedicle screw system with a multi-axial receiving member or bracket. In this system, an elongated stabilizer rod is compressed by a securing member including a compression cap and a threaded nut for bearing engagement directly against an underlying pedicle screw. However, one drawback of this system is that all of the components are stacked atop each other. Particularly, within the receiving member, the securing member is disposed on top of the stabilizing rod, which is in turn disposed on top of the pedicle screw. This results in an assembled system having a significant vertical stack-up dimension which can lead to post-surgical irritation of muscle and other patient tissue.

U.S. Pat. No. 6,187,005 describes a pedicle screw system in which a stabilizer rod is supported within a housing or bracket element at a position offset to one side of the pedicle screw. However, this system requires two separate securing members for respectively and independently fixating the pedicle screw and stabilizer rod. This use of multiple securing members undesirably increases surgical complexity and the time required to perform the surgical procedure.

In view of the foregoing, it would be desirable to provide improved pedicle screw systems and associated methods for spinal fixation.

### SUMMARY OF THE INVENTION

Embodiments of the present invention relate to provisional locking pedicle screw systems and associated methods for surgery. In various embodiments, the systems and methods described herein may be used to fuse together, mechanically immobilize, and/or adjust the alignment of adjacent vertebrae of the spine.

In an aspect of the present invention, an apparatus for attachment to bone is provided that includes a bone screw including a head (e.g., part-spherical head) and a shank, an anchor bracket, and a provisional locking member. The anchor bracket may include a cavity for receiving the head of the bone screw, a lower bore (e.g., part-circular void) beneath the cavity for receiving the shank of the bone screw, an upper bore (e.g., threaded upper bore) for receiving a securement member (e.g., set screw or component configured to form a press-fit connection with the upper bore), and an upwardly open channel disposed in a laterally offset relation to the cavity and the lower bore and configured to receive and support an elongated stabilizer rod. The provisional locking member may protrude from an interior wall of the anchor bracket and into the cavity, such that before the securement member is secured within the upper bore of the anchor bracket, the provisional locking member is configured to exert a frictional force against the head of the bone screw that semi-rigidly fixes the anchor bracket and the bone screw in place relative to each other. Despite this frictional force, the anchor bracket may be configured to rotate about the bone screw responsive to exertion of a mechanical force by a user (e.g., surgeon). Advantageously, this may allow the surgeon to adjust the provisionally-locked, relative positions of the pedicle screw and anchor bracket, for example, to accommodate the relative positions of one or more (e.g., two) additional pedicle screw/anchor bracket assemblies also in communication with the elongated stabilizer rod.

In some embodiments, the upper bore for receiving the securement member may be located at least partially above the cavity and the lower bore, such that securing the securement member within the upper bore more rigidly fixes the anchor bracket and the bone screw in place relative to each other. For example, in some embodiments, a central axis of the upper bore extends between the lower bore and the upwardly open channel, such that securing the securement member within the upper bore fixes the anchor bracket in place relative to the bone screw and the elongated stabilizer rod.

In some embodiments, the provisional locking member may include a bottom surface that is configured to contact the head of the pedicle screw, where the bottom surface (e.g., part-spherical surface) is substantially complimentary to a surface of the head of the bone screw. For example, in some embodiments, the provisional locking member may include a tab part fixed to a rear, interior wall of the anchor bracket and a semi-circular part in communication with the tab. The semi-circular part may be configured substantially in the shape of the letter “C”. The semi-circular part may form the bottom surface of the provisional locking member and may include two arms having free ends that extend into the cavity of the anchor bracket. When the two arms are placed into contact with the head of the bone screw, a distance between the two arms may increase in order to allow passage of the screw head into the cavity of the anchor bracket.

In still other embodiments, the provisional locking member may include an elongate rod fixed on at least one end to a side, interior wall of the anchor bracket, such that the elongate rod extends through the cavity of the anchor bracket. When the elongate rod is placed into contact with the head of the pedicle screw, the elongate rod may deform in order to allow passage of the head of the pedicle screw into the cavity of the anchor bracket. In some embodiments, the elongate rod may be fixed relative to the anchor bracket (e.g., by welding) and may remain within a patient's body after surgery. In other embodiments, the elongate rod may be removable from the anchor bracket by sliding the elongate rod outwardly in the direction of a central axis of the rod.

In some embodiments, the anchor bracket may additionally include a third bore located at least partially below the upwardly open channel and configured for placement over the head of the bone screw. Still further, in some embodiments, the anchor bracket may include a channel in communication with the third bore and the lower bore. The channel may be configured to retain the head of the bone screw within the interior of the anchor bracket when the shank of the bone screw is passed from the third bore to the lower bore through the channel.

In another aspect of the present invention, a method for bone fixation is provided that includes fixing a bone screw to a bone and provisionally locking an anchor bracket to the bone screw to semi-rigidly fix the relative positions of the bone screw and the anchor bracket in place. The method may further include placing an elongated stabilizer rod within the anchor bracket in a laterally offset relation to the bone screw and more rigidly (e.g., finally) locking the relative positions of bone screw, the anchor bracket, and the elongated stabilizer rod in place by securing a securement member to the anchor bracket.

In still another aspect of the present invention, an anchor bracket is provided that is configured to form a press-fit connection with the head of a bone screw. A method for manufacturing the anchor bracket is also provided.

In another aspect of the present invention, a multiple-component set screw is provided that includes a first component including a threaded region and a second component configured for matable attachment to the first component. In some embodiments, the second component may additionally include a first bottom surface (e.g., part-spherical surface) that is substantially complimentary to a surface of the bone screw and/or a second bottom surface (e.g., part-cylindrical surface) that is substantially complimentary to a surface of the elongated stabilizer rod.

### BRIEF DESCRIPTION OF THE DRAWINGS

For a better understanding of the present invention, including the various objects and advantages thereof, reference is made to the following detailed description, taken in conjunction with the accompanying illustrative drawings, in which like reference characters refer to like parts throughout, and in which:

FIG. 1A is a side, transparent view of a provisional locking pedicle screw system in accordance with some embodiments of the present invention;

FIG. 1B is a perspective view of the provisional locking member of FIG. 1A;

FIG. 2A is a perspective view of the pedicle screw system of FIG. 1A showing a provisional lock between an anchor bracket and a pedicle screw;

FIG. 2B is bottom view of the anchor bracket and provisional locking member of FIG. 1A;

FIG. 2C is front, transparent view of the anchor bracket and provisional locking member of FIG. 1A;

FIG. 2D is top view of the anchor bracket and provisional locking member of FIG. 1A;

FIG. 3 is a perspective view showing the addition of a stabilizer rod and a set screw to the assembly of FIG. 2A to provide a non-provisional lock;

FIGS. 4A and 4B are perspective, transparent views of a pedicle screw system with another type of provisional locking member according to some embodiments of the present invention;

FIGS. 4C and 4D are bottom and top views, respectively, of the anchor bracket and provisional locking member of FIGS. 4A and 4B;

FIGS. 5A-I are perspective views of an anchor bracket configured to form a press-fit connection with the head of a pedicle screw, and a method of manufacturing the anchor bracket, according to some embodiments of the present invention;

FIG. 6A-6B are perspective views of a two-piece set screw according to some embodiments of the present invention; and

FIG. 6C is a side, transparent view of a pedicle screw system that includes the two-piece set screw of FIGS. 6A-6B according to some embodiments of the present invention.

### DETAILED DESCRIPTION OF THE INVENTION

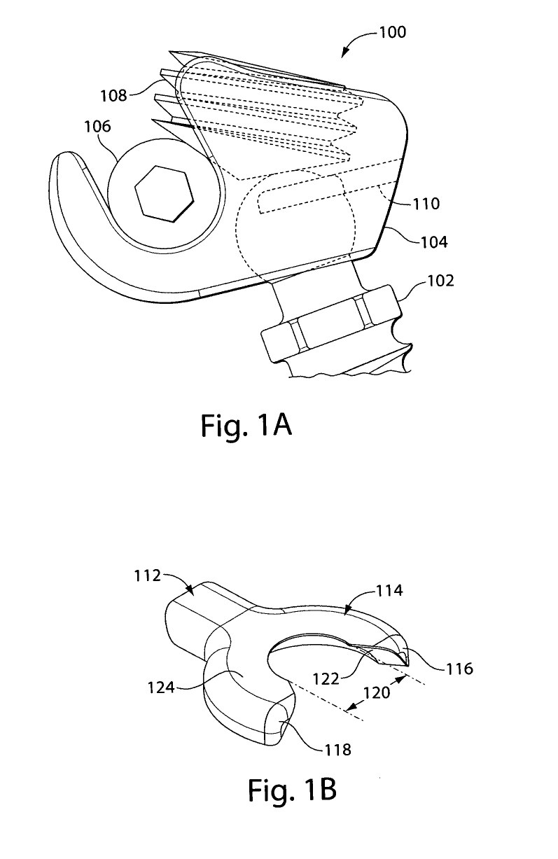

FIG. 1A is a side, transparent view of a provisional locking pedicle screw system 100 in accordance with some embodiments of the present invention. System 100 includes pedicle screw 102, anchor bracket 104, stabilizer rod 106, set screw 108, and provisional locking member 110. Provisional locking member 110 semi-rigidly fixes pedicle screw 102 to anchor bracket 104 before set screw 108 is screwed fully and tightened into place. This semi-rigid fixation allows anchor bracket 104 to rotate about pedicle screw 102, albeit in a controlled manner and only when manipulated mechanically (e.g., manually) by a user (e.g., surgeon). Advantageously, this may increase the ease with which a surgeon can assemble system 100, for example, by allowing the surgeon to semi-rigidly fix the relative positions of pedicle screw 102 and anchor bracket 104 prior to adding stabilizer rod 106 and set screw 108 to the assembly.

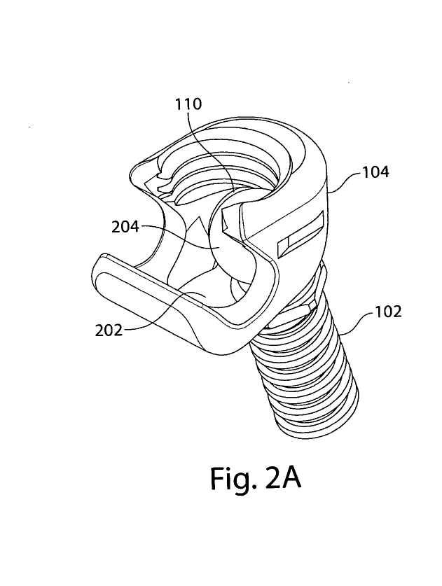

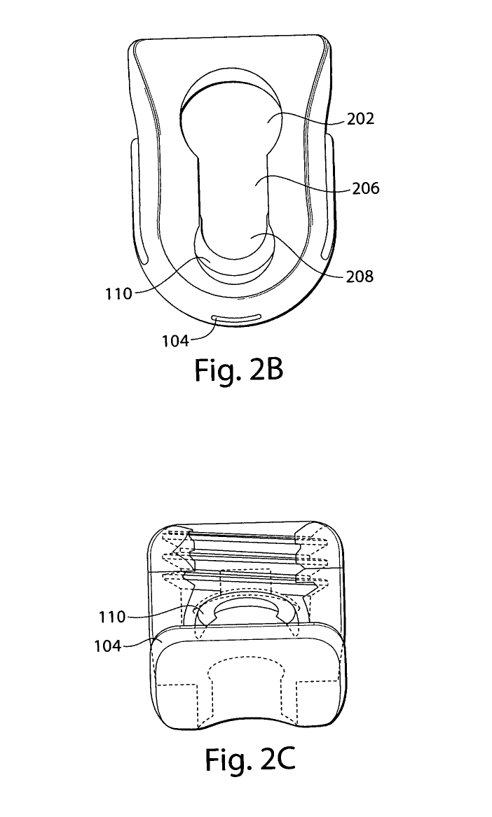

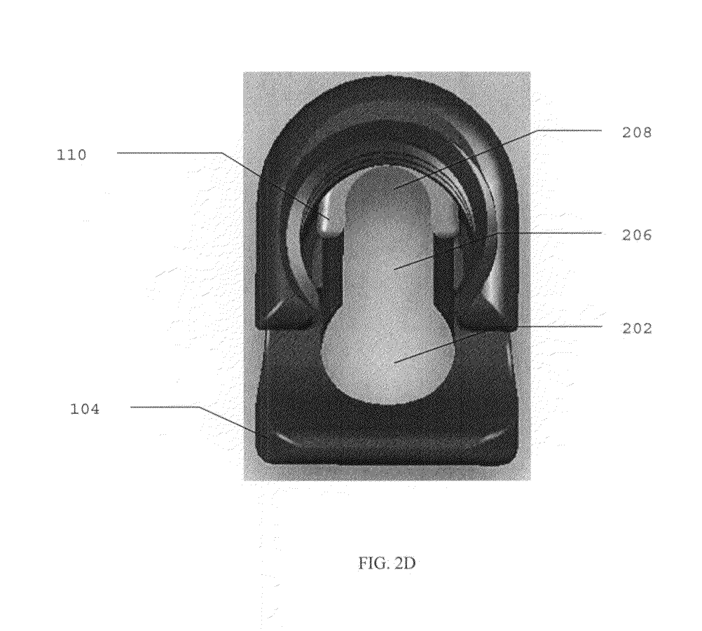

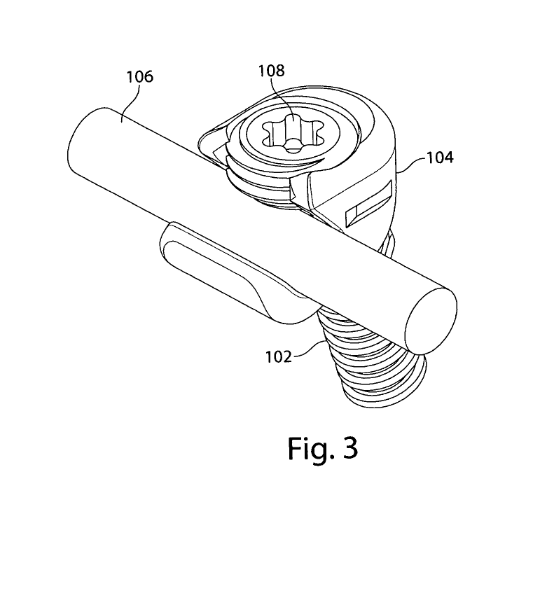

Once stabilizer rod 106 is positioned within anchor bracket 104, and further adjustments (if any) are made to the relative positions of pedicle screw 102 and anchor bracket 104, set screw 108 may be screwed fully into anchor bracket 104. This fixes the relative positions of bone screw 102, anchor bracket 104, and stabilizer rod 106 more rigidly within the assembly, which is also referred to herein as a non-provisional (e.g., final) lock. Prior to achieving the non-provisional lock, it may be necessary to adjust the provisionally-locked, relative positions of pedicle screw 102 and anchor bracket 104, for example, to accommodate the relative positions of one or more (e.g., two) additional pedicle screw/anchor bracket assemblies also in communication with stabilizer rod 106. In some embodiments, pedicle screw 102, anchor bracket 104, stabilizer rod 106, and set screw 108 may be substantially similar to, if not the same as, the corresponding components of the pedicle screw systems described in commonly-owned U.S. application Ser. No. 11/308,544, filed Apr. 4, 2006, which is hereby incorporated by reference herein in its entirety. Additional details regarding pedicle screw 102, anchor bracket 104, stabilizer rod 106, and set screw 108 are provided below in connection with the description of FIGS. 2A-3.

FIG. 1B is a perspective view of the provisional locking member of FIG. 1A according to some embodiments of the present invention. Provisional locking member 110 may include tab part 112 and semi-circular part 114 configured substantially in the shape of the letter “C”. Semi-circular part 114 may include opposed arcuate arms 116 and 118 separated at their free ends by distance 120. Semi-circular part 114 may additionally include bottom (e.g., part-spherical) surface 122 and top (e.g., flat) surface 124 that forms a part-circular bore (also referred to as a void). In the embodiment of FIG. 1B, provisional locking member 110 is a one-piece component of solid construction. In other embodiments, provisional locking member 110 may include multiple components fixed together by press fitting, heat fusion, soldering, a biocompatible adhesive, or according to any other suitable fixation approach or combination of approaches. Provisional locking member 110 may be made from a biocompatible material or any suitable combination of biocompatible materials including, for example, biocompatible metal(s) (e.g., titanium or steel), biocompatible polymer(s) (e.g., UHMWPE), and biocompatible ceramic, such as the doped silicon nitride ceramic disclosed in commonly-owned U.S. Pat. No. 6,881,229, which is hereby incorporated by reference herein in its entirety. Manufacturing provisional locking member 110 from a biocompatible material may allow member 110 to remain within the patient's body even after the surgery is complete.

With reference to FIGS. 1A and 1B, at least a portion of tab part 112 may fit within a corresponding recess located in a rear wall of anchor bracket 104. Tab part 112 may be fixed to anchor bracket 104 according to any suitable fixation approach or combination of approaches. In other embodiments, anchor bracket 104 and provisional locking member 110 may be a one-piece component of solid construction, with provisional locking member 110 and anchor bracket 104 forming part of the same, integral piece. Top surface 124 of provisional locking member 110 may be substantially parallel to a bottom surface of anchor bracket 104. Arms 116 and 118 of semi-circular part 114 may extend within a lower cavity of anchor bracket 104, which cavity may be configured to receive the head of pedicle screw 102. For example, the interior of anchor bracket 104 adjacent to the lower cavity may form a part-spherical surface configured for complimentary contact with a part-spherical head of pedicle screw 102.

When pedicle screw 102 is seated within the lower cavity of anchor bracket 104, arcuate arms 116 and 118 of semi-circular part 114 may extend around at least a substantial portion (e.g., greater than or equal to 180 degrees) of the circumference of pedicle screw 102. Bottom surface 122 of semi-circular part 114 may be substantially complimentary (e.g., part-spherical) to the surface of pedicle screw 102, for example, to maximize surface contact. At least a portion of the head of pedicle screw 102 may extend though the bore formed in top surface 124 of provisional locking member 110.

FIG. 2A is a perspective view of the pedicle screw system of FIG. 1A, in which provisional locking member 110 forms a provisional lock between pedicle screw 102 and anchor bracket 104. In some embodiments, anchor bracket 104 may be placed over pedicle screw 102 after pedicle screw 102 is at least partially screwed into a bone. With reference to FIGS. 1B and 2A, the provisional lock may be established by placing bore 202 formed within the bottom surface of anchor bracket 104 over head 204 of pedicle screw 102, and then sliding anchor bracket 104 laterally (e.g., parallel to the bottom surface of anchor bracket 104) to cause the free ends of arcuate arms 116 and 118 of provisional locking element 110 to contact and receive screw head 204. The force of screw head 204 against the free ends of arcuate arms 116 and 118 may cause an increase in the distance 120 between the arcuate arms, thereby allowing passage of screw head 204 into the seat of anchor bracket 104. Once head 204 is seated within anchor bracket 104, distance 120 between arcuate arms 116 and 118 may return to its original, reduced configuration. Provisional locking element 110 may exert downward and lateral forces against screw head 204 that semi-rigidly fix the relative positions of screw head 204 and anchor bracket 104.

FIGS. 2B, 2C, and 2D, respectively, are bottom, front, and top views of anchor bracket 104 and provisional locking member 110. Referring to FIGS. 2B and 2D, the bottom surface of anchor bracket 104 may form bore 202 (e.g., part-circular void) for receiving and permitting passage of head 204 of pedicle screw 102. Thus, a diameter of bore 202 may be slightly greater than a diameter of screw head 204. Bore 202 may be located beneath the channel formed in anchor bracket 104 for receiving stabilizer rod 106. The bottom surface of anchor bracket 104 may also form channel 206 in communication with bore 202 and lower bore 208 in communication with channel 206. Lower bore 208 may be formed within the seat of anchor bracket 104 configured to receive and retain screw head 204. Thus, a diameter of bore 208 may be greater than a diameter of the shank of pedicle screw 102 but slightly less than the diameter of screw head 204. Similarly, the width of channel 206 may be greater than the diameter of the screw shank but less than the diameter of screw head 204, to retain screw head 204 while permitting passage of pedicle screw 102 from bore 202 to bore 208.

FIG. 3 is a perspective view showing the addition of stabilizer rod 106 and set screw 108 to the assembly of FIG. 2A to convert the provisional lock to a non-provisional lock. As described above, the relative positions of pedicle screw 102, anchor bracket 104, and stabilizer rod 106 may be more rigidly fixed once set screw 108 is tightened into anchor bracket 104. Referring to FIGS. 1A and 3, set screw 108 contacts both pedicle screw 102 and stabilizing rod 106 in the non-provisionally locked configuration. Specifically, set screw 108 cooperates with the walls of anchor bracket 104 that contact pedicle screw 102 and stabilizing rod 106 to exert frictional forces on screw 102 and rod 106. In some embodiments, one or more of screw head 204, stabilizing rod 106, and the walls of anchor bracket 104 may include texturing (e.g., roughening) to increase these frictional forces. In the non-provisionally locked configuration, stabilizing rod 106 does not contact pedicle screw 102.

The following provides additional details regarding pedicle screw 102, anchor bracket 104, stabilizing rod 106, and set screw 108 according to some embodiments of the present invention. In some embodiments, two or more pedicle screw assemblies may be provided for attachment to at least a pair of adjacent patient bones such as spinal vertebrae, to maintain the skeletal structures in spaced relation while promoting bone ingrowth and fusion. Each assembly may include a pedicle screw 102, anchor bracket 104, provisional locking member 110, and set screw 108. Additionally, the pedicle screw assemblies may be connected together with a biocompatible stabilization member such as an elongated, generally cylindrical stabilizer rod 106 in order to provide a strong mechanical load bearing structure. Stabilizer rod 106 may run adjacent to the axis of the spine, lateral of or offset to the spinous processes, and medial of the transverse processes. In some embodiments, autologous bone or other suitable graft or fusion material may be placed between the adjacent vertebrae in an attempt to fuse the adjacent vertebrae together. Stabilizer rod 106 may be locked relative to the multiple anchor brackets 104 by the respective locking set screws 108. By coupling stabilizer rod 106 between multiple pedicle screw assemblies (which are in turn secured respectively to different, typically adjacent vertebrae), stabilizer rod 106 effectively limits or precludes intervertebral motion.

Generally, each pedicle screw 102 may include a threaded shank portion for engaging and securely anchoring to patient bone. The threads may have a self-tapping feature to allow for quicker insertion into the bone. Screw head 204 may include a drive feature such as a hex recess for receiving a drive tip of a suitable installation tool to facilitate insertion of screw 102 into the bone. Each pedicle screw 102 may be manufactured from a high strength bio-compatible material or combination of materials, allowing for load carrying capabilities. For example, in some embodiments, all components of pedicle screw system may be made from the same high strength biocompatible material or combination of materials such as, for example, titanium, steel, and the doped silicon nitride described in above-incorporated U.S. Pat. No. 6,881,229. Screw head 204 may be located proximally to the threaded portion of pedicle screw 102 and may have a generally spherical shape. When screw head 204 is positioned within the lower cavity of anchor bracket 104, screw head 204 may fit and articulate with a part-spherical seat of anchor bracket 104. This part-spherical seat may form lower bore 208 of anchor bracket 104, through which the elongated threaded shank of pedicle screw 102 may extend. As shown in FIG. 1A, an upper margin of screw head 204 may be below an upper margin of anchor bracket 104 when screw head 204 is seated within the lower cavity of anchor bracket 104.

Anchor bracket 104 also defines a trough or channel for receiving, supporting, and retaining the stabilizer rod 106. As shown in FIGS. 1A and 2A, this trough is defined by an arm or wing disposed laterally or in offset relation to one side of the lower bore and lower cavity of anchor bracket 104. Additionally, anchor bracket 104 includes an upper internally threaded bore positioned for at least partially overlying both screw head 204 (when screw head 204 is seated within the lower cavity) as well as the trough for receiving stabilizer rod 106. As shown in FIG. 1A, a vertical centerline through this upper threaded bore in anchor bracket 104 may extend generally between lower bore 208 and the trough formed in anchor bracket 104.

The threaded upper bore formed in anchor bracket 104 may receive a locking or securement member such as set screw 108. When set screw 108 is threaded into the upper bore, set screw 108 may have a lower face seated against and compressively engaging both stabilizer rod 106 and pedicle screw head 204. Accordingly, set screw 108 may create a friction or compression lock between anchor bracket 104 and stabilizer rod 106, while at the same time creating a friction or compression lock between bracket 104 and head 204 of pedicle screw 102. The upper bore may be a laterally open-sided, part-circular bore having a sufficient circumferential span (e.g., greater than 180 degrees) for receiving and retaining set screw 108. As shown in FIGS. 1A and 3, the open side of the upper bore is presented toward and generally partially overlies the trough for receiving stabilizer rod 106, which causes a portion of set screw 108 to overlie the trough and thus engage and lock with stabilizer rod 106 once the rod is seated within the assembly and set screw 108 is screwed fully into anchor bracket 104.

In some embodiments, the threads of set screw 108 and/or the corresponding threads of the upper bore of anchor bracket 104 may be configured to prevent screw 108 from backing out of the threaded upper bore. Alternatively or additionally, an upper side of set screw 108 may have a drive feature such as a hex-shaped recess formed therein for receiving a tool tip of a suitable installation tool to allow sufficient torque to be applied to screw 108. As set screw 108 is threadably advanced into the threaded upper bore of anchor bracket 104, a lower or underside face of set screw 108 engages and presses against stabilizer rod 106 seated within the associated trough of bracket 104. As such, set screw 108 may force rod 106 against the trough of anchor bracket 104 in a generally diametric direction corresponding with a contact point between set screw 108 and rod 106. Furthermore, set screw 108 may engage and press against head 204 of pedicle screw 102, thereby forcing head 204 against the part-spherical seat that forms lower bore 208 of anchor bracket 104. This may non-provisionally lock stabilizer rod 106 and pedicle screw 102 in place relative to anchor bracket 104.

Thus, the assembled system 100 (FIGS. 1A and 3) may provide a low implantation profile for reducing or eliminating post-operative patient discomfort attributable to tissue irritation. This low implantation profile may be due, at least in part, to the laterally-spaced configuration of pedicle screw 102 and stabilizer rod 106. In addition, pedicle screw system 100 may be adapted for surgical implantation via a surgical incision of minimal size, since all of the components may be installed and accessed from a common direction. That is, each pedicle screw 102 and associated anchor bracket 104 may be installed by access from above, followed by similar installation of stabilizer rod 106 quickly and easily into the upwardly open trough of each anchor bracket 104. Then, locking screws 108 may be similarly installed by access from above where, for each pedicle screw assembly, a single set screw 108 engages and locks both pedicle screw 102 and stabilizer rod 106 in place relative to the associated anchor bracket 104.

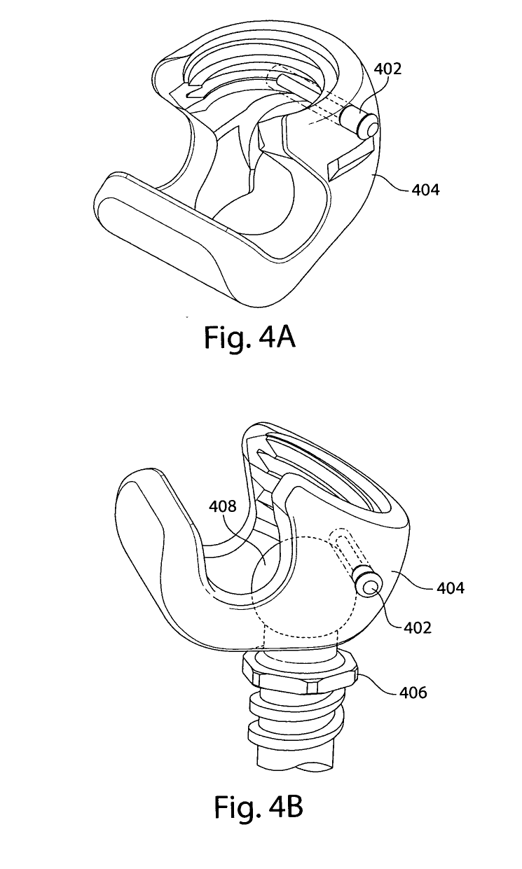

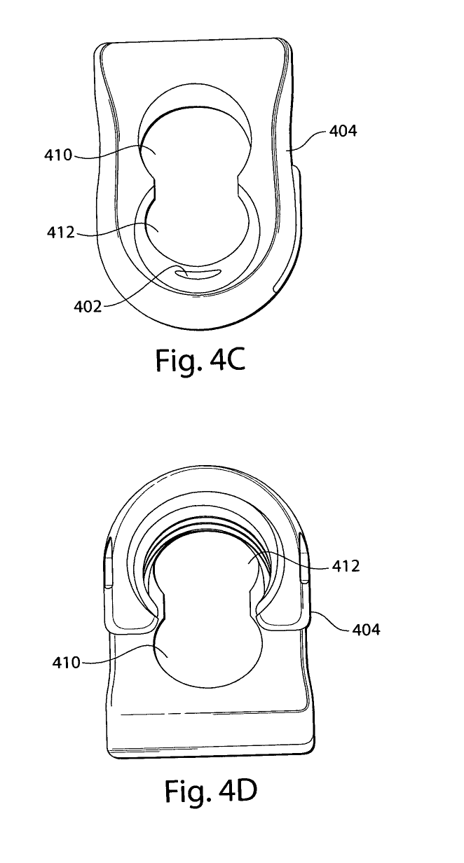

FIGS. 4A and 4B are perspective views of another type of provisional locking member 402 for use within a pedicle screw system according to some embodiments of the present invention. Generally, provisional locking member 402 includes an elongate rod that extends from one side wall of anchor bracket 404 towards an opposed side wall of bracket 404. Provisional locking member 402 may be located above the lower bore formed in anchor bracket 404 for receiving pedicle screw 406 including screw head 408, and below the upper bore formed in bracket 404 for receiving a set screw. In some embodiments, a central axis of provisional locking member 402 may be substantially transverse to a central axis of the lower bore formed in anchor bracket 404. Alternatively or additionally, provisional locking member 402 may extend through the lower cavity of anchor bracket 404 configured to receive the screw head.

FIGS. 4C and 4D, respectively, are bottom and top views of anchor bracket 404 and provisional locking member 402. The bottom surface of anchor bracket 404 may form bore 410 (e.g., part-circular void) for receiving and permitting passage of screw head 408. Thus, a diameter of bore 410 may be slightly greater than a diameter of screw head 408. Bore 410 may be located beneath the channel formed in anchor bracket 404 for receiving the stabilizer rod. The bottom surface of anchor bracket 404 may also form lower bore 412, which may intersect bore 410. This is in contrast to anchor bracket 104 in FIG. 2B, in which bores 202 and 208 are separated by channel 206. Thus, in some embodiments, anchor bracket 404 may have a reduced dimension (e.g., width from front to back) relative to anchor bracket 104. Lower bore 412 may be formed within the seat of anchor bracket 404 that is configured to receive and retain screw head 408. Thus, a diameter of bore 412 may be greater than a diameter of the shank of pedicle screw 406 but slightly less than the diameter of screw head 408. Seating pedicle screw head 408 within the lower cavity of anchor bracket 404 may involve placing pedicle screw head 408 through bore 410, and then sliding pedicle screw head into position within the seat of anchor bracket 404. When head 408 of pedicle screw 406 contacts provisional locking member 402, the rod may deform slightly (e.g., at least partially upwardly and/or rearwardly) to allow passage of screw head 408 into the seat that forms the lower bore. Once screw head 408 is seated within anchor bracket 404, frictional forces exerted by provisional locking member 402 and the walls of anchor bracket 404 adjacent to screw head 408 may semi-rigidly fix the relative positions of pedicle screw 406 and anchor bracket 404. The pedicle screw system of FIGS. 4A-D may be similar to, if not the same as, the pedicle screw system shown in FIGS. 1A-3 in all other respects.

In the embodiment of FIGS. 4A and 4B, provisional locking member 402 is a one-piece component of solid construction. In other embodiments, provisional locking member 402 may include multiple components fixed together by press fitting, heat fusion, soldering, a biocompatible adhesive, or according to any other suitable fixation approach or combination of approaches. Provisional locking member 402 may be made from any suitable material or combination of materials including, for example, biocompatible metal(s) (e.g., titanium or steel), biocompatible polymer(s) (e.g. UHMWPE), and biocompatible ceramic (e.g., doped silicon nitride). In some embodiments, provisional locking mechanism 402 may remain within a patient's body after the surgery is complete. In other embodiments, provisional locking mechanism may be removed from the pedicle screw assembly, for example, after a set screw is tightened into anchor bracket 404 and prior to closure of the patient. Provisional locking mechanism 402 may be removed from and/or reinserted to anchor bracket 404, for example, by sliding (e.g., pulling) an end of the rod outwardly in the direction of the central axis of the rod.

In FIGS. 4A and 4B, one end of provisional locking member 402 may fit within a corresponding recess in a side wall of anchor bracket 404, whereas the other end of member 402 may be free. In other embodiments, both ends of provisional locking member 402 fit within corresponding recesses in the first side wall and second side wall, respectively, of anchor bracket 404. The end(s) of provisional locking member 402 may be fixed to anchor bracket 404 according to any suitable fixation approach or combination of approaches (e.g., welding). In other embodiments, anchor bracket 404 and provisional locking member 402 may be a one-piece component of solid construction, with provisional locking member 402 and anchor bracket 404 forming part of the same, integral piece. FIGS. 1-4D are only illustrative and combinations and modifications of these embodiments are within the scope of the present invention. For example, in some embodiments, a pedicle screw system may be provided in which provisional locking member 402 is housed within anchor bracket 104 (FIG. 1) (e.g., in place of provisional locking member 110). In other embodiments, a pedicle screw system may be provided in which provisional locking member 110 (e.g., c-shaped member) is housed within anchor bracket 404 (e.g., in place of provisional locking member 402).

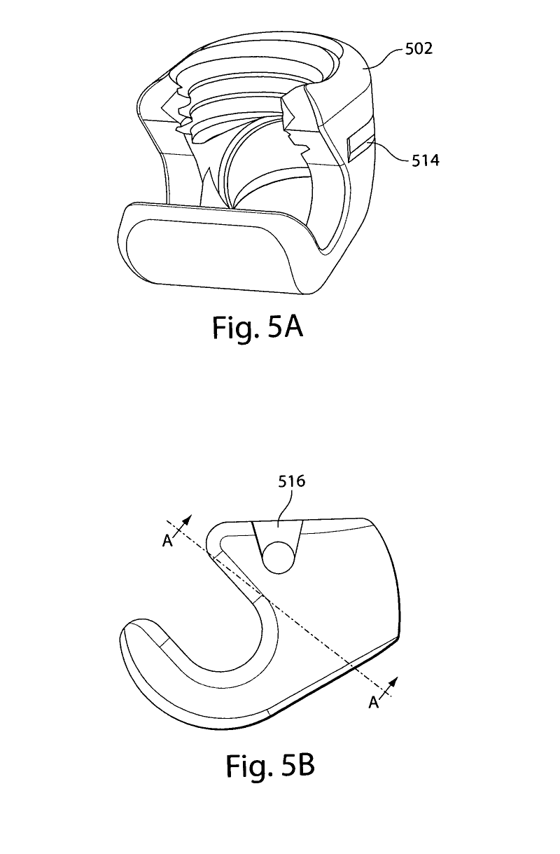

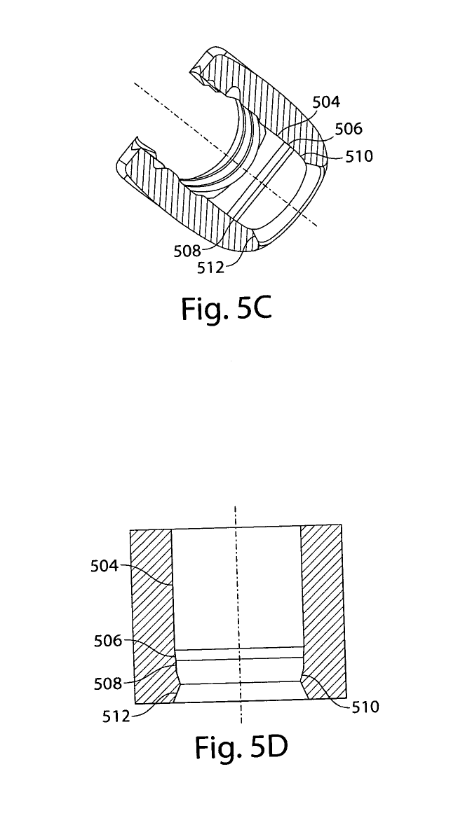

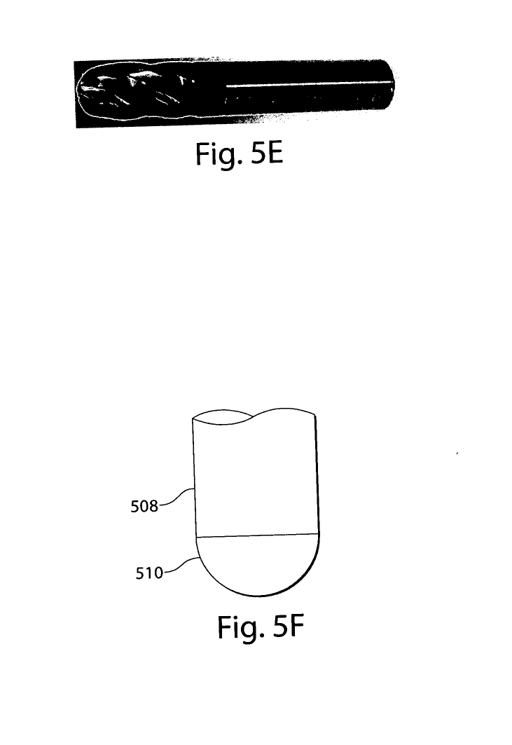

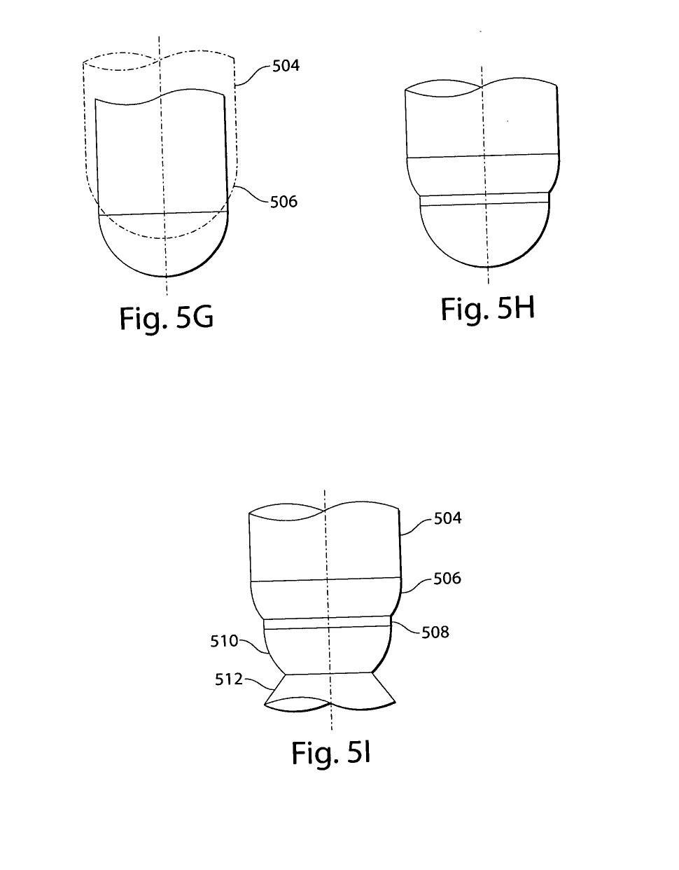

FIG. 5A is perspective view of another embodiment of an anchor bracket 502 according lo to the present invention. Generally, the surfaces (504-512; FIGS. 5C-5D) within anchor bracket 502 for receiving a pedicle screw (e.g., pedicle screw 102) form a press-fit connection with the head of the pedicle screw. The press fit occurs when the screw head is seated within the surface of the anchor bracket that forms the lower bore. The press-fit mechanism may semi-rigidly lock the relative positions of the pedicle screw and anchor bracket 502 before a set screw is tightened into bracket 502. Anchor bracket 502 may be used alone or in combination with at least one of provisional locking member 110 (FIGS. 1A and 1B) and provisional locking member 402 (FIGS. 4A and 4B). In various embodiments, the bottom configuration of anchor bracket 502 may be similar to, if not the same as, the configuration shown in FIG. 2B or FIG. 4C. Anchor bracket 502 may include channel 514 configured for interaction with a tool for inserting bracket 502 into a patient's body.

FIG. 5B is a side view of the anchor bracket of FIG. 5A, albeit that the embodiment of FIG. 5B includes a different configuration 516 for interacting with an insertion tool. FIG. 5C is a perspective view of the anchor bracket taken along lines A-A in FIG. 5B. As shown, the anchor bracket includes surfaces 504, 506, 508, 510, and 512. FIG. 5D is a reoriented, simplified view of surfaces 504-512 of anchor bracket 502. Generally, surface 508 forms a narrow ring or lip that forms the press-fit with the head (e.g., part-spherical head) of the pedicle screw.

FIGS. 5E-I illustrate a method of manufacturing the anchor bracket shown in FIGS. 5A-D according to an embodiment of the present invention. FIG. 5E shows a mill with a generally part spherically-shaped end and a cylindrical shank, for use in cutting a part-spherical, part-cylindrical bore into the anchor bracket. This mill may be used, for example, when the head of the pedicle screw is at least partially spherical. In other embodiments, the mill (e.g., the tip of the mill) may have other configurations (e.g., part-conical) in order to compliment other configurations of the head of a pedicle screw. As shown in FIG. 5F, a first end mill is driven into the anchor bracket to an appropriate depth, in order to form part-cylindrical surface 508 and part-spherical surface 510. The diameter of the first end mill is slightly smaller than a diameter of the pedicle screw, which creates a frictional, press-fit that limits the movement of the screw when the screw head is inserted or forced into the region formed by surfaces 508 and 510. Next, a second end mill is driven into the anchor bracket along the same axis of insertion of the first end mill, in order to form part-cylindrical surface 504 and part-spherical surface 506 (FIG. 5G). The second end mill is not driven as deep as the first end mill, such that at least a portion of surface 508 remains intact. The diameter of the second end mill is larger than that of the first end mill and the pedicle screw head, which allows the screw head to slide freely into and out of the region formed by surfaces 504 and 506. Surface 512 is created as the final cut which controls the amount of screw angulation.

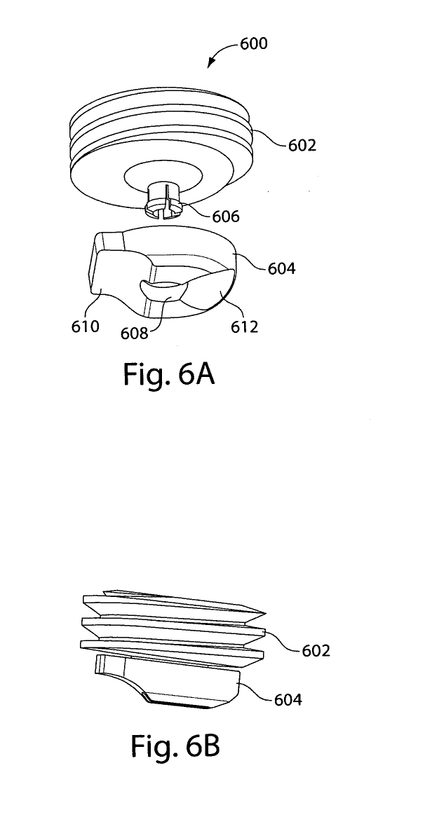

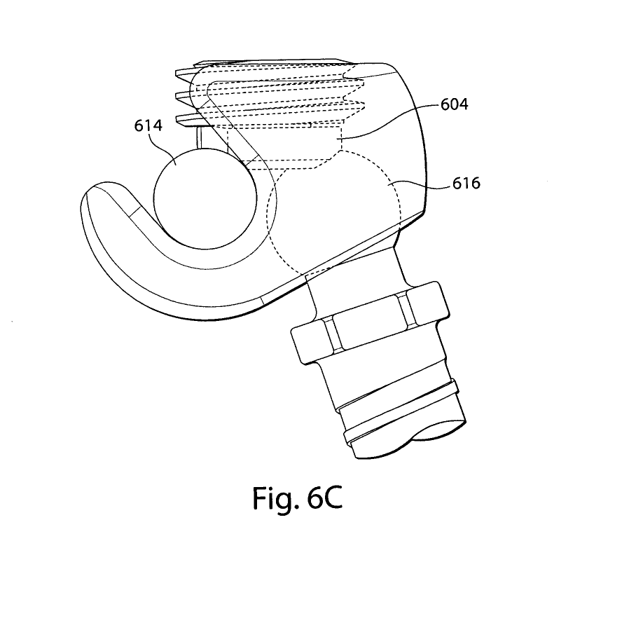

FIGS. 6A-6B are perspective views of a set screw 600 that includes two components 602 (e.g., top component) and 604 (e.g., bottom component) according to some embodiments of the present invention. Component 602 may be threaded and may include, for example, three threads for engaging the threaded upper bore of an anchor bracket. Component 602 may also include protrusion 606 for matably attaching to recess 608 formed within component 604, for example, according to a press-fit connection. Component 604 may be unthreaded and may include bottom surfaces 610 and 612 for contacting a stabilizer rod and the head of a pedicle screw, respectively. For example, bottom surface 610 may have a shape (e.g., part-cylindrical) that is complimentary to the shape of the stabilizer rod. Bottom surface 612 may have a shape (e.g., part-spherical) that is complimentary to the shape of the head of the pedicle screw. The connection between components 602 and 604 may allow components 602 and 604 to rotate axially with respect to one another, thereby allowing for centering (e.g., self-centering or centering with the aid of a user) of component 604 with respect to the stabilizer rod and screw head when component 604 is placed into contact with these components.

FIG. 6C is a side, transparent view of a pedicle screw system that includes the two-piece set screw of FIGS. 6A and 6B according to some embodiments of the present invention. As shown, the two-piece set screw 600, and more specifically component 604 of set screw 600, provides increased contact area for contacting rod 614 and screw head 616. This increases the frictional forces that can be exerted against rod 614 and screw head 616 by the set screw to fix these components in place. In various embodiments, the pedicle screw system of FIG. 6C may be similar to, if not the same as, the pedicle screw system shown in FIGS. 1A-3, FIGS. 4A-D, or FIGS. 5A-B, in all other respects.

Thus it is seen that pedicle screw systems and associated methods for surgery are provided. Although particular embodiments have been disclosed herein in detail, this has been done by way of example for purposes of illustration only, and is not intended to be limiting with respect to the scope of the appended claims, which follow. In particular, it is contemplated that various substitutions, alterations, and modifications may be made without departing from the spirit and scope of the invention as defined by the claims. Other aspects, advantages, and modifications are considered to be within the scope of the following claims. The claims presented are representative of the inventions disclosed herein. Other, unclaimed inventions are also contemplated. The applicant reserves the right to pursue such inventions in later claims.

What is claimed is:

1. Apparatus for attachment to bone, the apparatus comprising:
a bone screw comprising a head and a shank; and
an anchor bracket comprising:
a cavity for receiving the head of the bone screw;
a lower bore beneath the cavity for receiving the shank of the bone screw;
an upper bore for receiving a securement member;
an upwardly open channel disposed in a laterally offset relation to the cavity and the lower bore and configured to receive and support an elongated stabilizer rod; and
a provisional locking member protruding from an interior wall of the anchor bracket and into the cavity, wherein before the securement member is secured within the upper bore, the provisional locking member is configured to exert a frictional force against the head of the bone screw that semi-rigidly fixes the anchor bracket and the bone screw in place relative to each other.

2. The apparatus of claim 1, wherein despite the frictional force the anchor bracket is configured to rotate about the bone screw responsive to exertion of a mechanical force by a user.

3. The apparatus of claim 1, wherein the upper bore is located at least partially above the cavity and the lower bore, such that securing the securement member within the upper bore more rigidly fixes the anchor bracket and the bone screw in place relative to each other.

4. The apparatus of claim 3, wherein a central axis of the upper bore extends between the lower bore and the upwardly open channel, such that securing the securement member within the upper bore fixes the anchor bracket in place relative to the bone screw and the elongated stabilizer rod.

5. The apparatus of claim 4, wherein the upper bore comprises a threaded upper bore and the securement member comprises a set screw.

6. The apparatus of claim 1, further comprising the securement member and the elongated stabilizer rod.

7. The apparatus of claim 1, wherein the provisional locking member comprises a bottom surface configured to contact the head of the bone screw and wherein the bottom surface is substantially complimentary to a surface of the head of the bone screw.

8. The apparatus of claim 7, wherein bottom surface is part-spherical.

9. The apparatus of claim 7, wherein the provisional locking member further comprises:
a tab part fixed to a rear, interior wall of the anchor bracket; and
a semi-circular part in communication with the tab, the semi-circular part forming the bottom surface of the provisional locking member and comprising two arms having free ends that extend into the cavity of the anchor bracket.

10. The apparatus of claim 9, wherein when the two arms are placed into contact with the head of the bone screw, a distance between the two arms increases in order to allow passage of the head of the bone screw into the cavity of the anchor bracket.

11. The apparatus of claim 1, wherein the provisional locking member comprises an elongate rod fixed on at least one end to a side, interior wall of the anchor bracket, such that the elongate rod extends through the cavity of the anchor bracket.

12. The apparatus of claim 11, wherein when the elongate rod is placed into contact with the head of the bone screw, the elongate rod deforms in order to allow passage of the head of the bone screw into the cavity of the anchor bracket.

13. The apparatus of claim 1, wherein the anchor bracket further comprises:
a third bore located at least partially below the upwardly open channel and configured for placement over the head of the bone screw.

14. The apparatus of claim 13, wherein the anchor bracket further comprises:
a channel in communication with the third bore and the lower bore and configured to retain the head of the bone screw within the interior of the anchor bracket when the shank of the bone screw is passed from the third bore to the lower bore through the channel.

15. Apparatus for attachment to bone, the apparatus comprising:
a bone screw comprising a head and a shank; and
an anchor bracket comprising:
a cavity for receiving the head of the bone screw;
a lower bore beneath the cavity for receiving the shank of the bone screw;
an upper bore for receiving a securement member;
an upwardly open channel disposed in a laterally offset relation to the cavity and the lower bore and configured to receive and support an elongated stabilizer rod; and
an interior surface adjacent to the lower bore and configured to form a press-fit connection with the head of the bone screw.

16. Apparatus for attachment to bone, the apparatus comprising:
a bone screw comprising a head and a shank;
an elongated stabilizer rod;
a multi-component set screw comprising a first component with a threaded region and a second component configured for matable attachment to the first component; and
an anchor bracket comprising:
a cavity for receiving the head of the bone screw;
a lower bore beneath the cavity for receiving the shank of the bone screw;
an upper bore for receiving a multi-component set screw; and
an upwardly open channel disposed in a laterally offset relation to the cavity and the lower bore and configured to receive and support the elongated stabilizer rod.

17. The apparatus of claim 16, wherein the second component of the multi-component set screw comprises:
a first bottom surface that is substantially complimentary to a surface of the bone screw; and
a second bottom surface that is substantially complimentary to a surface of the elongated stabilizer rod.

18. A method for bone fixation, the method comprising:
fixing a bone screw to a bone;
provisionally locking an anchor bracket to the bone screw to semi-rigidly fix the relative positions of the bone screw and the anchor bracket in place;
placing an elongated stabilizer rod within the anchor bracket in a laterally offset relation to the bone screw; and
more rigidly locking the relative positions of bone screw, the anchor bracket, and the elongated stabilizer rod in place by securing a securement member to the anchor bracket.

19. The method of claim 18, wherein provisionally locking the anchor bracket to the bone screw comprises exerting a frictional force against a head of the bone screw, wherein despite the frictional force the anchor bracket is configured to rotate about the head of the bone screw responsive to exertion of a mechanical force by a user.

20. The method of claim 19, wherein exerting a frictional force comprises contacting the head of the bone screw with a bottom surface of a member that protrudes from an interior wall of the anchor bracket, wherein the bottom surface is substantially complimentary to a surface of the head of the bone screw.

21. The method of claim 19, wherein exerting a frictional force comprises:
placing the head of the bone screw into contact with opposed arms of a member configured substantially in the shape of the letter “C”; and
exerting a lateral force on the two arms with the head of the bone screw, wherein the lateral force causes a distance between the two arms to increase in order to allow passage of the head of the bone screw into a cavity of the anchor bracket.

22. The method of claim 19, wherein contacting the head of the bone screw with the member comprises:
placing the head of the bone screw into contact with an elongate rod; and
exerting a lateral force on the elongate rod with the head of the bone screw, wherein the lateral force causes the elongate rod to deform in order to allow passage of the head of the bone screw into a cavity of the anchor bracket.

23. The method of claim 18, wherein securing a securement member to the anchor bracket comprises:
matably attaching a first set screw component comprising threads to a second set screw component, wherein the second set screw component comprises first and second surfaces that are complimentary to the surfaces of the bone screw and the elongated stabilizer rod, respectively;
contacting the surfaces of the bone screw and the elongated stabilizer rod with the first and second surfaces of the second set screw component; and
screwing the first set screw component into the anchor bracket.

24. The method of claim 18, wherein provisionally locking an anchor bracket to the bone screw comprises:
placing a first bore located in a bottom surface of the anchor bracket over a head of the bone screw; and
moving the anchor bracket laterally relative to the bone screw through a channel formed in the bottom surface of the anchor bracket, wherein the channel is in communication with the first bore and a second bore and is configured to retain the head of the bone screw within the interior of the anchor bracket when the shank of the bone screw is passed from the first bore to the second bore through the channel.

25. Apparatus for attachment to bone, the apparatus comprising:
means for fixing a bone screw to a bone;
means for provisionally locking an anchor bracket to the bone screw to semi-rigidly fix the relative positions of the bone screw and the anchor bracket in place;
means formed in the anchor bracket for receiving an elongated stabilizer rod in a laterally offset relation to the bone screw; and
a securement member for more rigidly locking the relative positions of bone screw, the anchor bracket, and the elongated stabilizer rod in place.
